(19) United States
(12) Patent Application Publication
(10) Pub. No.: US 2003/0009153 A1
(43) Pub. Date: Jan. 9, 2003

(54) Ultrasonic enhancement of drug injection

(75) Inventors: Axel F. Brisken, Fremont, CA (US); Robert Zuk, Atherton, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pharmasonics, Inc., Sunnyvale, CA (US)

(21) Appl. No.: 10/228,904

(22) Filed: Aug. 26, 2002

Related U.S. Application Data
(63) Continuation of application No. 09/364,616, filed on Jul. 29, 1999.
Application No. 09/364,616 is a continuation-in-part of application No. 09/255,290, filed on Feb. 22, 1999.
Application No. 09/364,616 is a continuation-in-part of application No. 09/126,011, filed on Jul. 29, 1998, now Pat. No. 6,464,680.

(51) Int. Cl. A61B 17/00

(52) U.S. Cl. 604/890.1; 604/22; 600/459

(57) ABSTRACT

A method of enhancing cellular absorption of a substance delivered into a target region of a patient's body, comprising: (a) delivering the substance to the target region; and (b) directing vibrational energy to the target region, wherein the vibrational energy is of a type and in an amount sufficient to enhance absorption into cells of the target region.

### CROSS-REFERENCES TO RELATED APPLICATIONS

This application is a continuation of application Ser. No. 09/364,616 (Attorney Docket No. 017148-001820), filed Jul. 29, 1999, which was a continuation-in-part of application Ser. Nos. 09/126,011 (Attorney Docket No. 017148-001810), filed on Jul. 29, 1998, and Ser. No. 09/255,290 (Attorney Docket No. 017148-001800), filed on Feb. 22, 1999, the full disclosures of which are incorporated herein by reference.

### BACKGROUND OF THE INVENTION

1. Field of the Invention

The present invention relates to methods and devices for enhancing cellular absorption of a substance delivered into a target region of a patient's body. A current standard technique for the delivery of drugs or other substances into the human body is needle injection. A bolus containing the drug is typically injected into muscle or fatty tissue and is then absorbed into the interstitial fluid or directly into the fatty tissue. Over a period of time, the vascular system of the body takes over and flushes the drug out of the interstitial fluid or fat and into the capillaries. From there, the cardiovascular system widely distributes the drug into the rest of the patient's body.

Newly developed drugs often have application only to specific organs or sections of organs. As such, systemic distribution of the drug throughout the remainder of the body can: (1) dilute very expensive drugs, weakening their effects, (2) generate an effect systemically instead of locally, and (3) widely distribute a drug which may be toxic to other organs in the body. Furthermore, some of the newly developed drugs include DNA in various forms, such DNA being degraded very rapidly by natural mechanisms in the body if delivered systemically, thus preventing a full dose from reaching the designated organ. Accordingly, it would be desirable to provide devices, kits, and methods for delivering such site-specific drugs in a manner which enhances absorption specifically at the site of their delivery into a target region of a patient's body.

2. Description of the Background Art

Catheters and methods for intravascular transfections are described in U.S. Pat. No. 5,328,470 and published in PCT applications WO 97/12519; WO 97/11720; WO 95/25807; WO 93/00052; and WO 90/11734. See also copending application Ser. No. 09/223,231, the full disclosure of which is incorporated herein by reference.

Ultrasound-mediated cellular transfection is described or suggested in Kim et al. (1996) Hum. Gene Ther. 7:1339-1346; Tata et al. (1997) Biochem. Biophy. Res. Comm. 234:64-67; and Bao et al. (1997) Ultrasound in Med. & Biol. 23:953-959. The effects of ultrasound energy on cell wall permeability and drug delivery are described in Harrison et al. (1996) Ultrasound Med. Biol. 22:355-362; Gao et al. (1995) Gene Ther. 2:710-722; Pohl et al. (1993) Biochem. Biophys. Acta. 1145:279-283; Gambihler et al. (1994) J. Membrane Biol. 141:267-275; Bommannan et al. (1992) Pharma. Res. 9:559-564; Tata and Dunn (1992) J. Phys. Chem. 96:3548-3555; Levy et al. (1989) J. Clin. Invest. 83:2074-2078; Feschheimer et al. (1986) Eur. J. Cell Biol. 40:242-247; and Kaufman et al. (1977) Ultrasound Med. Biol. 3:21-25. A device and method for transfection of endothelial cells suitable for seeding vascular prostheses are described in WO 97/13849.

Local gene delivery for the treatment of restenosis following intravascular intervention is discussed in Bauters and Isner (1998) Progr. Cardiovasc. Dis. 40:107-116 and in Back and March (1998) Circ. Res. 82:295-305.

### BRIEF SUMMARY OF THE INVENTION

The present invention provides methods, devices, and kits for enhancing cellular absorption of a drug or other substance into a local target region of a patient's body, thereby avoiding the undesirable effects of the substance being widely dispersed throughout the patient's body by the patient's cardiovascular system. By “cellular absorption,” it is meant that at least a significant proportion of the total amount of drug delivered to the site is absorbed or otherwise taken up by the cells within or surrounding the target site. The nature of the cells will vary depending on the target site. The cells may be muscle or fat cells receiving transcutaneous, intraoperative, or percutaneous injection. In a first preferred aspect of the present invention, these cells comprise the patient's myocardial tissue. In a second preferred aspect of the present invention, the cells may comprise any solid tissue cell which is a target for gene transfection, particularly myocardial and other muscle tissues. The cells may also be endothelial, epithelial, and/or other cells which line the interior or exterior of target organs, or brain cells protected by the blood/brain barrier, or organ cells in general. Lastly, the cells may also be specific organ cells of a target organ.

Specifically, a method is provided for enhancing cellular absorption of a substance, comprising the steps of: (a) delivering the substance to the target tissue region, and (b) directing vibrational energy to the target region, wherein the vibrational energy is of a type and amount sufficient to enhance absorption of the substance into the cells of the target region. In a preferred aspect of the present invention, the vibrational energy has a mechanical index in the range of 0.1 to 20. Devices for emitting ultrasonic vibrations of a type and amount sufficient to enhance cellular absorption may comprise a wide variety of known transducer systems, such as piezoelectric, magnetostrictive or single crystal devices.

The application of such vibrational energy to the target region increases cellular absorption on the order of 3 to 300 times or more for biological reporters such as luciferase and beta-galactosidase genes and for drugs such as heparin, probucol, liposome-complexed plasmid DNA, cationic polymer complexed DNA, plus viral vectors including adeno-associated viral DNA, vascular endothelial growth factors, and naked DNA, relative to their uptake in the absence of the vibrational energy.

The present invention will be useful for delivering a wide variety of drugs, genes, and other therapeutic and/or diagnostic substances to target tissue sites. The substances will usually have a pharmological or biological effect and may range from those generally classified as small molecule drugs (usually below 2 kD, more usually below 1 kD), such as hormones, peptides, small nucleic acids, carbohydrates, and the like to those generally classified as large molecule drugs (usually above 500 kD, often above 50 kD, and sometimes above 200 kD) such as large proteins, complete regulatory and structural genes, large carbohydrates, and the like. The present invention will be particularly effective in delivering macromolecules such as biologically active proteins and nucleic acids. For delivery to the muscles in general, or the myocardium in particular, useful substances, proteins and the genes which encode such proteins, e.g., angiogenesis stimulators, such as angiogenic cytokines including vascular endothelial growth factor (VEGF) and basic fibroblast growth factor (BFGF). Other useful substances and genes include endothelial nitric oxide synthase (eNOS) for inhibiting restenosis; brain naturatic peptides; beta-adrenogenic receptors for preventing congestive heart failure; erythropoietin (EPO); clotting factors, such as Factor VIII and Factor IX; human growth hormone; insulin; interferons, particularly including interferon-A for treating neoplasms; interleukins; and various “secretory proteins” which are proteins that are secreted from transfected cells and exert biological effects on other cells and tissues. Such secretory proteins may include hepatocyte growth factor, atrial naturiuretic factor, VEGF, -1 antitrypsin, -Iduronidase, Iduronate-2-sulfatase, glucocerebrosidase, -glacuronidase and neurotrophin. For many or most of these, it will be preferred to introduce a gene which encodes the desired therapeutic protein together with any necessary regulatory nucleic acid sequences to a desired target tissue. By transfecting the target tissue with the therapeutic protein gene, the cells can then produce the therapeutic proteins in therapeutically effective amounts. Ultrasound in combination with DNA-based vaccines would enhance protein expression by improving the humoral and cellular immune response.

The delivery of nucleic acids (usually in the form of genes) to target cells is generally referred to as “transfection.” The methods of the present invention may be advantageously applied to cellular transfection of target tissues since they are capable of significantly increasing transfection efficiency, i.e. the amount of nucleic acid materials taken up by the muscle cells and cellular nuclei to which they are delivered. The methods of the present invention are useful with a wide variety of nucleic acid types. For example, it has been found that significant transfection efficiencies can be obtained even with naked DNA, i.e., nucleic acids which are not incorporated into liposomes, virosomes, viral vehicles, (eg: adenovirus, retrovirus, lentivirus, and adeno-associated virus), plasmids, or other conventional nucleic acid vehicles. The methods are not limited to such naked nucleic acids, however, they are also suitable for the delivery of nucleic acids incorporated into liposomes and cationic polymer complexes such as virosomes, vesicles; viral vectors, including both adenoviral vectors and retroviral vectors; plasmids, and the like.

In a preferred method, the substance is delivered to the target cells in the target tissue of the host. This delivery can be accomplished transcutaneously or percutaneously by way of an injection needle or needles, injected in high-velocity, small-volume jets of delivery fluid, or delivered interoperatively. The substance can also be delivered by a controlled release device such as a microsphere. Substance delivery could also be accomplished by positioning the distal end of a delivery device, (such as a catheter or hand held device), proximal to a target region of tissue, wherein a vibrational energy emitter is positioned at the distal end of the device. For delivery through the skin or surgical use, the device may be constructed similarly to a syringe having an ultrasonic driver on or near the needle tip. For internal delivery, the device will typically be formed as a catheter for intraluminal or endoscopic introduction to a target site.

By “delivery,” it is meant that the drug, gene, or other substance is injected or otherwise physically advanced into a target region of tissue. Injection can be performed with a needle and a pressurized source of the substance, e.g. a syringe. A controlled delivery device or depot containing the drug could also be implanted within the target tissue. Substances of interest will typically be delivered through the internal walls and membranes of organs (particularly the epicardium and endocardium when targeting the myocardium), blood vessels, and the like, as well as through the skin. In some instances, the catheter will be percutaneously introduced to a blood vessel or open body cavity in order to permit access to the internal organs and gene delivery sites.

The present invention also provides a device for enhancing cellular absorption of a substance delivered to a target tissue region of a patient's body comprising a substance delivery system and a vibrational energy emitter which is adapted to emit vibrational energy of a type and amount sufficient to enhance cellular absorption in the tissue. Preferably, the substance delivery system comprises one or more injection needles.

In one embodiment, the injection needle and the ultrasound energy emitter form a small integrated device which is received at the distal end of a catheter. In one aspect of this embodiment, the energy emitter may include one or more vibrational energy emitting transducers received within the injection needle. In various embodiments, the vibrational energy emitter is disposed proximate to the substance delivery system. In certain preferred embodiments, the vibrational energy emitter is mounted directly to the injection needle. The vibrational energy emitter may also be disposed concentrically around the substance delivery system. Specifically, the substance delivery system may comprise an injection needle with the vibrational energy emitter mounted directly on the injection needle. In various embodiments, the injection needle system comprises a plurality of retractable radially extending injection needles which are positioned at the distal end of a catheter such that when the catheter is received into an intraluminal cavity, the injection needles can be radially extended outward puncturing the wall of the cavity and entering into the underlying tissue. In various preferred embodiments, the vibrational energy emitter emits vibrational energy laterally outward in radial directions away from the distal end of a catheter such that the catheter can be positioned in parallel orientation to the target tissue, such as when the distal end of the catheter is received in a blood vessel or other luminal cavity.

Also, specific embodiments of the present invention may include additional diagnostic, measurement, or monitoring components or capabilities. For example, the device for emitting vibrational energy to the target region may be adapted to detect the net electromechanical impedance of the target tissue in opposition with the vibrational device thus enabling an operator to determine when the distal end of the device contacts the target tissue by observing a change in the effective impedance of the device. Moreover, an echo ranging transducer positioned on the distal end of the catheter or other device can be used to determine the thickness and condition of the target tissue. This can be accomplished by operating the ranging transducer in a pulse echo mode, and characterizing the amplitude, spectral content, and timing of the returning echoes. Furthermore, an electrocardiograph monitoring electrode can optionally be positioned on the distal end of the catheter adjacent the substance delivery system for monitoring potentials in a patients myocardium. This can be useful for providing therapy to the site in the myocardium which is responsible for rhythm abnormalities.

Kits according to the present invention may comprise the delivery devices in combination with instructions for use setting forth any of the above-described methods.

### BRIEF DESCRIPTION OF THE DRAWINGS

FIG. 1 shows a microscopic pictorial representation of a substance being injected into a target tissue by way of an injection needle.

FIG. 2 is a microscopic pictorial representation of cellular absorption of the substance of FIG. 1 mediated by ultrasound energy.

FIG. 3A is a sectional view of the distal tip of a device for enhancing cellular absorption of a substance delivered to a region of target tissue.

FIG. 3B is a sectional view of the distal tip of an alternate device for enhancing cellular absorption of a substance.

FIG. 3C is a sectional view of the distal tip of a third alternative device for enhancing cellular absorption of a substance.

FIG. 4 is a pictorial view of the device of FIGS. 3A, 3B or 3C received into a ventricle of a patient's heart.

FIG. 5A is an enlarged pictorial view of the device of FIGS. 3A, 3B, 3C, and 4 shown penetrating through a patient's endocardium into the patient's myocardium.

FIG. 5B is an enlarged pictorial view of the device of FIGS. 3A, 3B, 3C, and 4 shown penetrating through the wall of a patient's coronary artery and into the patient's myocardium.

FIG. 5C is an enlarged pictorial view of the device of FIGS. 3A, 3B, 3C, and 4 shown penetrating through a patient's epicardium and into the patient's myocardium, via an open thoracotomy surgical approach.

FIG. 5D is an enlarged pictorial view of needle injection of a drug with transesophageal ultrasonic enhancement.

FIG. 5E is an enlarged pictorial view of an alternate system of needle injection of a drug with remote, simultaneous ultrasonic enhancement.

FIG. 6A is a side elevation sectional view of a first embodiment of the vibrational energy emitter of the device of FIG. 3A.

FIG. 6B is a side elevation sectional view of a combined vibrational energy emitter and injection device of FIG. 3B.

FIG. 7 is a side elevation sectional view of an alternative embodiment of the vibrational energy emitter of FIG. 6A.

FIG. 8 is a side elevation sectional view of the vibrational emitter of FIG. 6A with an echo ranging transducer and electrophysiology electrode at its distal end.

FIG. 9 is a representation of the electromechanical impedance magnitude of a vibrational energy emitter in contact with fluid and with the myocardium.

FIG. 10 is a representation of the electromechanical impedance phase angle of a vibrational energy emitter in contact with fluid and with the myocardium.

FIG. 11 is a pictorial representation of the range finding transducer emitting a signal into the myocardium.

FIG. 12 is a representation of the return echo from the range finding transducer's emitted pulse, shown coming back from the myocardium.

FIG. 13 is a pictorial representation of an alternative embodiment of a device for enhancing cellular absorption having a vibrational energy emitter and plurality of radially outwardly extending retractable injection needles.

FIG. 14 is a pictorial representation of a patient's leg immersed in a fluidic environment which is subjected to vibrational energy.

FIG. 15 is an illustration of a kit comprising devices for enhancing cellular absorption and instructions for its use.

FIG. 16 is an illustration of treatment of soft tissue lesions by combined needle injection and ultrasonic emission.

FIG. 17 illustrates an ultrasound transducer device that was employed in the examples described in the Experimental section.

FIG. 18 is a graph referred to in the Experimental section showing enhanced transfection with ultrasonic treatment in a rabbit model.

FIG. 19 is a graph referred to in the Experimental section showing enhanced blood flow with VEGF and ultrasonic treatment in ischemic hind limbs in young rabbits.

FIG. 20 is an illustration of the individual test results used to generate FIG. 19.

FIG. 21 is a graph referred to in the Experimental section showing enhanced blood flow with VEGF and ultrasonic treatment in ischemic hind limbs in older rabbits.

FIG. 22 is a graph referred to in the Experimental section showing enhanced blood pressure ratio with VEGF and ultrasonic treatment in ischemic hind limbs in young rabbits.

FIG. 23 is an illustration of the individual test results used to generate FIG. 22.

FIG. 24 is a graph referred to in the Experimental section showing enhanced blood pressure ratio with VEGF and ultrasonic treatment in ischemic hind limbs in older rabbits.

FIG. 25 is a graph referred to in the Experimental section showing enhanced angiographic score with VEGF and ultrasonic treatment in ischemic hind limbs in young rabbits.

FIG. 26 is an illustration of the individual test results used to generate FIG. 25.

FIG. 27 is a graph referred to in the Experimental section showing enhanced angiographic score with VEGF and ultrasonic treatment in ischemic hind limbs in older rabbits.

FIG. 28 is a graph referred to in the Experimental section showing the effect of ultrasonic pretreatment, prior to DNA injection.

### DETAILED DESCRIPTION OF THE INVENTION

The present invention provides a method for enhancing cellular absorption of a drug, gene, or other substance into a local target region of a patient's body, thereby avoiding the undesirable effects of the substance being widely dispersed throughout the patient's body by the patient's cardiovascular system or having the substance compromised by the natural cleansing activities of the patients organs, as follows.

First, the substance is delivered to the target region of a patient's tissues. Secondly, vibrational energy is directed to the target region, wherein the vibrational energy is of a type and amount sufficient to enhance absorption of the substance into the cells of the target region, as will be explained.

The methods, systems, and kits of the present invention will be suitable for delivering virtually any therapeutic, diagnostic, or other substance where it is desired that the substances be taken up by individual cells comprising part of a human or other animal tissue mass. As a first general example, the substances may be therapeutic drugs, proteins, small molecules, or the like, where the drugs are intended to enter through the cell walls to have a desired therapeutic or other effect. In a second general example, the substances will be nucleic acids intended to transfect the target cells in the tissue mass. Such nucleic acids which may delivered by the methods and devices of the present invention will comprise nucleic acid molecules in a form suitable for uptake into target cells within a host tissue, usually smooth muscle cells lining the blood vessels, or skeletal cells or cardial muscle. The nucleic acids will usually be in the form of bare DNA or RNA molecules, where the molecules may comprise one or more structural genes, one or more regulatory genes, antisense strands, strands capable of triplex formation, or the like. Commonly, such nucleic acid constructs will include at least one structural gene under the transcriptional and translational control of a suitable regulatory region. Optionally, but not necessarily, the nucleic acids may be incorporated in a viral, plasmid, or liposome vesicle delivery vehicle to improve transfection efficiency.

If viral delivery vehicles are employed, they may comprise viral vectors, such as retroviruses, adenoviruses, and adeno-associated viruses, which have been inactivated to prevent self-replication but which maintain the native viral ability to bind a target host cell, deliver genetic material into the cytoplasm of the target host cell, and promote expression of structural or other genes which have been incorporated in the particle. Suitable retrovirus vectors for mediated gene transfer are described in Kahn et al. (1992) CIRC. RES. 71:1508-1517, the disclosure of which is incorporated herein by reference. A suitable adenovirus gene delivery is described in Rosenfeld et al. (1991) SCIENCE 252:431-434, the disclosure of which is incorporated herein by reference. Both retroviral and adenovirus delivery systems are described in Friedman (1989) SCIENCE 244:1275-1281, the disclosure of which is also incorporated herein by reference.

The nucleic acids may be present in a lipid delivery vehicle which enhances delivery of the genes to target smooth muscle cells within the vascular epithelia or elsewhere. Transfection with a lipid delivery vehicle is often referred to as “lipofection.” Such delivery vesicles may be in the form of a liposome where an outer lipid bilayer surrounds and encapsulates the nucleic acid materials. Alternatively, the nucleic complexes may be in the form of a nucleic acid-lipid dispersion, nucleic acid-lipid emulsion, or other combination. In particular, the complexes may comprise liposomal transfection vesicles, including both anionic and cationic liposomal constructs. The use of anionic liposomes requires that the nucleic acids be entrapped within the liposome. Cationic liposomes do not require nucleic acid entrapment and instead may be formed by simple mixing of the nucleic acids and liposomes. The cationic liposomes avidly bind to the negatively charged nucleic acid molecules, including both DNA and RNA, to yield complexes which give reasonable transfection efficiency in many cell types. See Farhood et al. (1992) Biochem. Biophys. Acta. 1111:239-246, the disclosure of which is incorporated herein by reference. A particularly preferred material for forming liposomal vesicles is lipofection which is composed of an equimolar mixture of dioleylphosphatidyl ethanolamine (DOPE) and dioleyloxypropyl-triethylammonium (DOTMA), as described in Felgner and Ringold (1989) Nature 337:387-388, the disclosure of which is incorporated herein by reference.

It is also possible to combine these two types of delivery systems. For example, Kahn et al. (1992), supra., teaches that a retrovirus vector may be combined in a cationic DEAE-dextran vesicle to further enhance transformation efficiency. It is also possible to incorporate nuclear proteins into viral and/or liposomal delivery vesicles to even further improve transfection efficiencies. See, Kaneda et al. (1989) Science 243:375-378, the disclosure of which is incorporated herein by reference.

The nucleic acids will usually be incorporated into a suitable carrier to facilitate delivery and release into the blood vessels according to the present invention. The carriers will usually be liquids or low viscosity gels, where the nucleic acids will be dissolved, suspended, or otherwise combined in the carrier so that the combination may be delivered through the catheter and/or carried by the catheter and released intravascularly at the treatment site. Alternatively, the nucleic acids may be provided in a dry or solid form and coated onto or otherwise carried by the catheter or the vibrational surface.

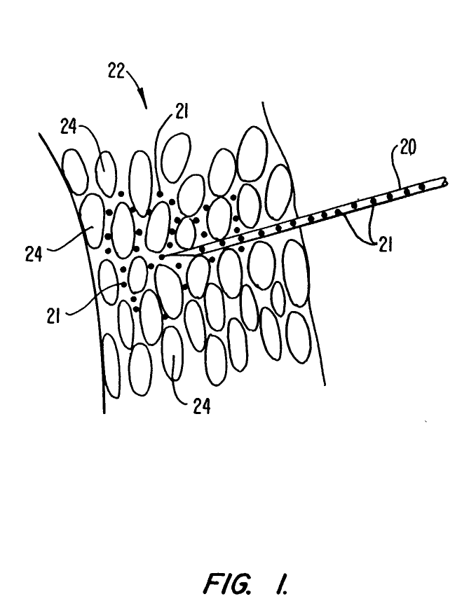

FIG. 1 illustrates an injection needle 20 delivering a drug or other substance 21 into a region of target tissue 22 which is comprised of a plurality of cells 24. In the absence of the present invention's application of vibrational energy, drug 21 will tend to absorb slowly into cells 24 causing the drug 21 to be distributed widely in the patient's body thus either diluting a very expensive drug and thereby weakening its effect or generating a systemic effect on the patient instead of the desired local effect.

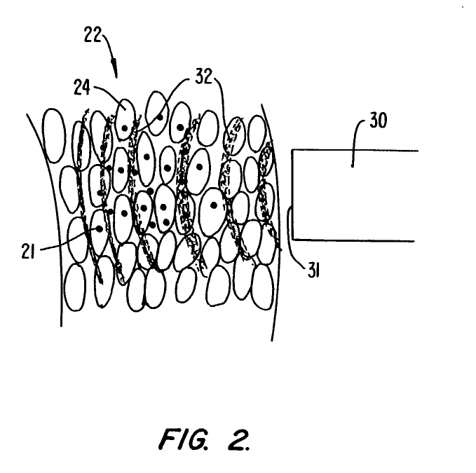

However, in accordance with the present invention as shown in FIG. 2, a vibrational emitter 30, may be used to emit ultrasound waves 32 into the target tissue 22 in a type and in an amount sufficient such that drug 21 is instead readily absorbed into cells 24. As will be explained herein, emitter 30 preferably has a vibrational energy with a mechanical index in the range of 0.1 to 20. In addition, this vibrational energy preferably has a frequency range of 20 kHz to 3.0 MHz, and more preferably in the range of 200 kHz to 1.0 MHz.

The bio-effects of ultrasonic energy are typically mechanical in nature (cavitational or pressure effects) or thermal in nature (heat due to absorption of energy or energy conversion). The American Institute for Ultrasound in Medicine (AIUM) and the National Electrical Manufacturers Association (NEMA) in “Standard for Real-Time Display of Thermal and Mechanical Indices on Diagnostic Ultrasound Equipment”, 1991, have together defined the term “mechanical index” for medical diagnostic ultrasound operating in the frequency range of 1 to 10 MHz, as follows.

Mechanical index, (hereafter “MI”), is defined as the peak rarefactional pressure (in MPa) at the point of effectivity (corrected for attenuation along the beam path) in the tissue divided by the square root of the frequency (in MHz), or

MI=P(MPa)/sqrt(f[MHz])

Typical ultrasound devices specify operating conditions based on frequency (kHz or MHz) and intensity (W/cm2). As defined above, MI effects embody frequency and intensity, and therefore for the purpose of this invention, ultrasound conditions will be specified solely in terms of MI.

The tolerated range for diagnostic imaging equipment is up to an MI of 1.9. MI values over 1 to 2 represent acoustic levels which can cause mechanical bio-effects including excessive membrane damage and cell necrosis due to inertial cavitation, microstreaming, or radiation pressure. In addition, MI's above specific diagnostic limits, (such as MI's in excess of 20), are typically regarded as potentially damaging to tissue, and are instead exploited by various therapeutic devices for tissue destruction, ablation, and deep heating.

Moreover, as the MI increases, the temperature elevation in the tissue will also tend to increase. Unfortunately, biological dangers also increase concurrent with excessive temperature elevation in the tissue. Specifically, a temperature increase in normal vascularized muscle tissue of more than 5 degrees Centigrade may cause unwanted formation and accumulation of clot. Moreover, a temperature elevation in the tissue of greater than 5 degrees can cause significant heating of the tissue resulting in denaturation and necrosis. Moreover, increased temperatures of tissue may cause inflammation in the area of treatment. Accordingly, temperature elevations within the tissue are typically kept 5 degrees or less to avoid such clotting, inflammation or other tissue damage.

Accordingly, in a preferred aspect of the present invention, ultrasound energy is employed within a “therapeutic window” wherein the range of ultrasound energy is generally above the level used for diagnostic purposes, yet below the level where profound tissue damage occurs.

In particular, in an aspect of the present invention, ultrasound conditions which favor a high mechanical index yet preferably produce only a low temperature elevation in the tissue are used to induce a preferred cellular response which promotes increased porosity and subsequent uptake of therapeutic agents. In accordance with the present invention, a preferred range for enhanced drug delivery is an MI of 0.1 to 20, and more preferably, a MI of 0.3 to 15, still more preferably a MI of 0.5 to 10, and most preferably a MI of 0.5 to 5. Preferably, the duty cycle of the transducer of the present invention is set such that the temperature elevation in the tissue remains less than about 5 degrees Celsius.

By virtue of controlled mechanical action on the tissue interfaces and cellular membranes, temporary disruption of membranes occurs, thereby increasing porosity and perfusion of adjacent liquids into cells. Ultrasound induced membrane disruption has moderate durability, with most cells returning to normal. In accordance with an aspect of the present invention, controlled disruption of membranes allows therapeutic agents to more readily pass into the cells and cell organelles including the nucleus. An advantage of this system is its improvement in the efficiency of gene transfection and subsequent expression of genes.

In contrast to the present invention, existing ultrasound transducers used for diagnostic purposes are typically highly damped, have low sensitivity, and have a broad bandwidth response. Such transducers are designed to generate very short ultrasound pulses and to receive highly complex and irregular return echoes which are used to generate images or other diagnostic information. Moreover, these transducers tend to generate a high temperature rise in the tissue when operated at a high duty cycle (i.e., the fraction of time during which the ultrasound field is energized) such as greater than 50%, but are incapable of generating a high MI, primarily because of their high operating frequency (3 MHz and above) and heavy damping. As the above equation for MI indicates, at constant pressure, MI decreases by the square root of frequency. Accordingly, these diagnostic device transducers cannot generate enough pressure (amplitude) to overcome the frequency related loss. These restrictions apply to transcutaneous as well as intravascular diagnostic ultrasound devices.

Ultrasound transducers used for therapeutic purposes generally fall into two categories: thermal devices having high frequency for thermal effects, i.e., deep heating, and mechanical devices having low frequency for mechanical effects, i.e., lithotripsy and clot lysis.

Thermal devices are used transcutaneously for deep heating and tissue destruction, invasively for destroying pathological tissue, and percutaneously for ablation. In contrast to the present invention, these devices operate at a high duty cycle (greater than 50%) and typically raise the tissue temperature on the order of at least 4 degrees Celsius. Similar to the existing ultrasonic diagnostic transducers, ultrasonic therapeutic transducers are generally incapable of operating with a high MI due to their high operating frequency and heavy damping.

Mechanical devices for lithotripsy, or disintegration of concretions within the body, are exclusively transcutaneous and not invasive. They operate at low frequency (20-500 kHz) and have very large acoustic apertures which allow the ultrasound energy to be focussed within the body. By virtue of their frequency and application, these devices operate with a high MI. Clot lysis using low frequency ultrasound energy is achieved by positioning a transducer external to the body and percutaneously transferring vibrational energy into veins and arteries through a translating wire coupled to the transducer. These devices suffer from high frictional power loss when used in curved arteries and lumens.

Transfection of mammalian cells in vitro was reported in Kim et al. (1996), supra. In that publication, the most efficient transfection of fibroblasts and chondrocytes was achieved with continuous exposure of 1 MHz ultrasonic energy of peak pressures up to 400 kPa. (ie: having an MI of 0.4). Employing an MI of greater than 0.4, such devices were found to fragment plasma DNA and therefore were not used in their transfection studies.

In contrast, the present invention can achieve gene transfection employing much higher MI's with higher peak pressures by setting its duty cycle sufficiently low enough to prevent DNA fragmentation induced by ultrasound. For example, as will be shown in Experiment Number one, employing a duty cycle of 6% with an MI of 1.8, a 24.5 times increase in beta-galactosidase transfection was achieved. In this way, higher transfection efficiencies can be achieved without significantly damaging the nucleic acids being delivered.

In the present invention, vibrational energy emitters capable of high MI yet maintaining low temperature increases in the tissue were coupled to a substance delivery device which can be used transcutaneously or intraoperatively in the form of a hand held probe or injection device, and percutaneously in the form of a catheter are provided.

In the present invention, the application of vibrational energy to the target region increases cellular absorption on the order of 3 to 300 times or more for drugs such as hormones, peptides, proteins, nucleic acids, genes, carbohydrates, DNA vaccines, and angiogenesis stimulators relative to their uptake in the absence of such vibrational energy. For example, cellular transfections (DNA transfer into the nucleus of the cell, as manifested by altered expression of the cell) of a reporter gene Beta galactosidase into muscle tissue has been shown to increase by over a factor of 25 at an MI of 0.05 to 5.0.

For cellular transfection in target muscles and other tissues, there is concern not only with damage to the tissue but also to the DNA/RNA structures being delivered. It has been observed (as described above) that high peak pressures, e.g., over 400 kPa at 1 MHz (ie: 0.4 MI), can have a deleterious effect on the DNA/RNA structures being delivered. In in vitro transfection the use of ultrasonic energy having lower peak pressures, however, is disadvantageous because of a potentially significant reduction in transfection efficiency. The present invention, in contrast, has recognized that higher mechanical indices (and higher peak pressures) can be utilized by employing a limited duty cycle, usually below 50%, more usually below 25%, typically in the range from 0.1% to 10%, more typically in the range from 1% to 10%. The duty cycle is defined as the percentage of time during which the transducer is active or energized. A 100% duty cycle represents a substantially continuous energy emission from the transducer. The duty cycle will generally be controlled by energizing and de-energizing the ultrasonic transducer at a fairly rapid rate, typically having a relatively short “burst” length, i.e., the length time for a single burst of vibrational energy. The on/off frequency will generally be referred to as the pulse repetition frequency (PRF), and the vibrational energy will usually be applied in short bursts of relatively high intensity (power) interspersed in relatively long periods of no excitation (or much lower excitation). Thus, for a 1% duty cycle, the energy will emanate 1% of the time, but frequently at a relatively rapid on/off rate, with exemplary PRF being in the range from 10 to 10,000, often from 100 to 5,000, and more often from 300 to 3,000 (usually expressed as Hz).

Broad, preferred, and exemplary values for each of the ultrasonic energy parameters are set forth below.

BROAD, PREFERRED, AND EXEMPLARY
TREATMENT CONDITIONS

| | Broad | Preferred | Exemplary |
|---|---|---|---|
| Mechanical Index (MI) | 0.1 to 20 | 0.3 to 15 | 0.5 to 5 |
| Frequency (kHz) | 100 to 5,000 | 300 to 3,000 | 500 to 1,500 |
| Duty Cycle (%) | 0.1 to 50 | 0.5 to 20 | 1 to 10 |
| Pulse Repetition Frequency | 10 to 10,000 | 100 to 5,000 | 300 to 3,000 |
| (Hz) | | | |

In preferred embodiments, emitter 30 has a generally planar vibrational surface 31 which is positioned proximate to, or engages, a surface of target tissue 22, such as the external surface of the patient's skin or an external surface of an organ comprising target tissue 22.

The step of delivering the substance 21 by way of injection needle 20 or otherwise can be accomplished either prior to, concurrently with, or after the step of directing vibrational energy emitted by emitter 30 to cells 24 of target region 22 as is shown in FIG. 2. In an alternative method, the step of delivering substance 21 into target region 22 can be accomplished by forming an incision in the patient's skin and depositing the substance 21 (e.g. in the form of an implantable release depot) into the incision.

In preferred embodiments, target region 22 can be the myocardium of the patient and substance 21 can be any substance which promotes angiogenesis, for example VEGF, BFBF, and the like, and their corresponding genes. It is to be understood, however, that target region 22 is illustrative of any target tissue region in the patient's body and substance 21 is illustrative of any of a variety of drugs or other substances which have a therapeutic effect upon a local region of the patient's tissues. For example, substance 21 can include any drug useful in the treatment of vascular diseases, including proteins, such as growth factors, clotting factors, clotting factor inhibitors; nucleic acids, such as the genes which encode the listed proteins, antisense genes, and other secretory proteins. It may also include chemotherapeutic agents for the treatment of cancer and other hyperproliferative diseases; and may also include vaccines and any other type of therapeutic substance or agent.

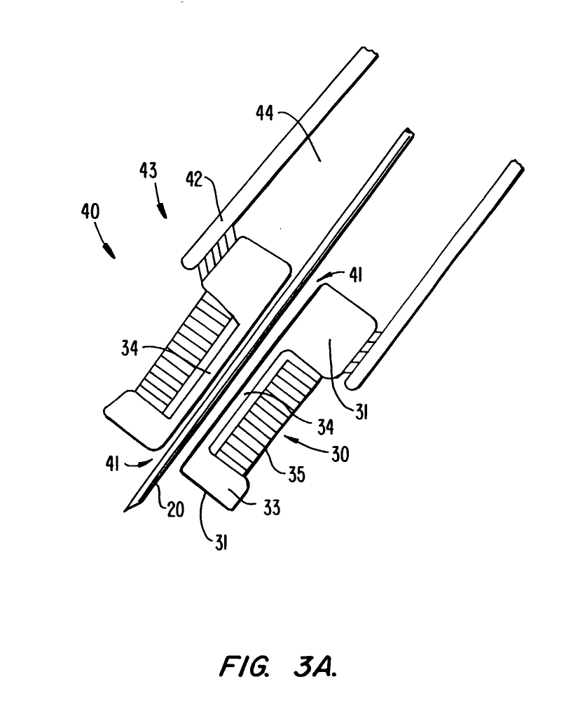

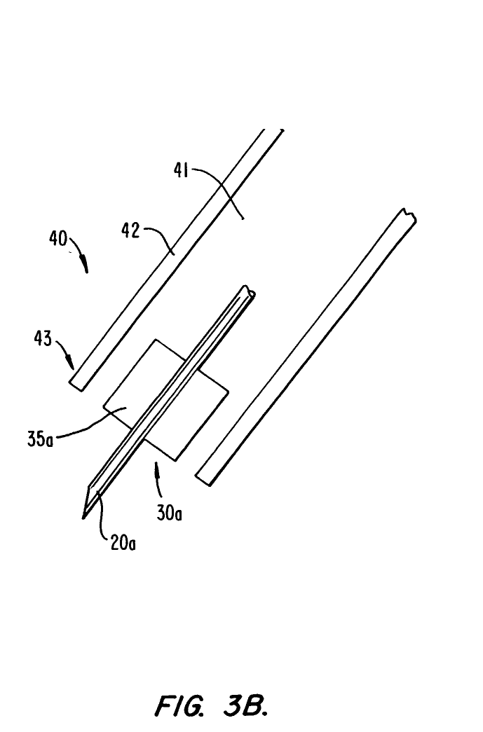

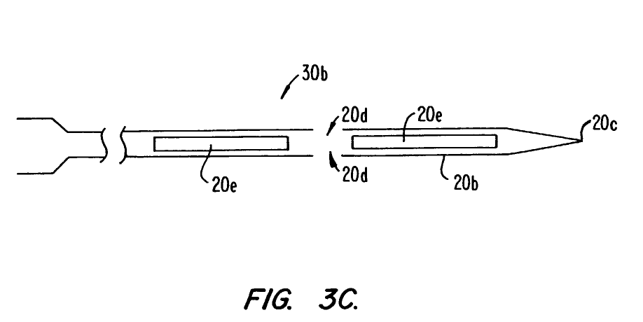

As is shown in FIG. 3A, the injection needle 20 of FIG. 1 and the vibrational energy emitter 30 of FIG. 2 can preferably be combined into an integrated device 40 which is preferably positioned at a distal end of a catheter 42. In a preferred embodiment, vibrational emitter 30 completely surrounds injection needle 20. An advantage of integrated device 40 is that a drug can be delivered to a target tissue region by way of injection needle 20 concurrently with the application of vibrational energy to the target region by emitter 30. As is seen in FIG. 3A, vibrational emitter 30 preferably comprises an inertial mass 31 and a head mass 33 with a piezostack 35 positioned therebetween. Inertial mass 31 and head mass 33 are preferably linked together by way of an internal rod 34 and the mass of radiating head 33 and the dimensions of piezostack 35 would be adjusted to achieve the final desired frequency and output displacement. Piezostack 35 would typically comprise on the order of twenty layers of ceramic material. Emitter 30 may alternatively include a piezoelectric tube, a magnetostrictive device, or transducer bars. Emission may be in either the forward or lateral directions.

Preferably, injection needle 20 is slidably received in lumen 44 of catheter 42 and in lumen 41 of emitter 30. As such, injection needle 20 can be easily advanced or withdrawn to project out of the distal end of emitter 30, as desired. For example, the distal end 42 of catheter 40 can first be safely introduced into a patient's body and positioned proximal a target tissue region. Subsequently, injection needle 20 can then be advanced to project out of the emitter 30 and into the target tissue, thereby delivering a drug or other substance to the target tissue.

FIG. 3B shows an alternative embodiment corresponding to FIG. 3A, but with the injection needle 20a being connected directly to energy emitter 30a. Energy emitter 30a may comprise a piezostack 35a. Vibration of piezostack 35a causes needle 20a to vibrate. Needle 20a, being short and stiff in nature, will oscillate axially at the same frequency and at the same amplitude of the piezostack 35a. The tip of the needle 20a will thus act as an ultrasound emitter. An advantage of this device is that, as energy emitter 30a is attached to the substance delivery injection needle 20a, very effective application of ultrasound energy at the exact point of drug delivery is achieved. The present needle 20a is preferably fabricated from stainless steel, while the emitter is fabricated from piezoelectric material. This system is an improvement over prior developments because the needle 20a will thus oscillate at the site of injection causing formation of microbubbles as the liquid agent is injected. The presence of microbubbles enhances cavitation which improves the efficiency of transfections. Needle 20a will thus act as an extension of transducer 30a with the tip of the needle vibrating at the same frequency and amplitude as that of the transducer. Similar to the device of FIG. 3a, the device of FIG. 3B is preferably received in the system of catheter 40, as previously described.

FIG. 3C shows a third alternative embodiment of a device for enhancing cellular absorption of a substance comprising an integrated injection needle and ultrasound energy emitter system 30b. System 30b comprises an injection needle 20b having a penetration tip 20c and one or more portals 20d. Penetration tip 20c preferably has a 14-30 gauge diameter for easy skin penetration. One or more internally mounted transducers 20e are provided. Preferably, transducers 20e are located proximal and distal or just proximal or just distal the locations of portal or portals 20d. Accordingly, drug injection can be provided such that ultrasonic energy emitted by transducers 20e is adjacent to the point of drug delivery through portals 20d, thus ensuring that ultrasound energy is applied directly to the target tissue, thereby increasing drug delivery effectiveness into the target cells.

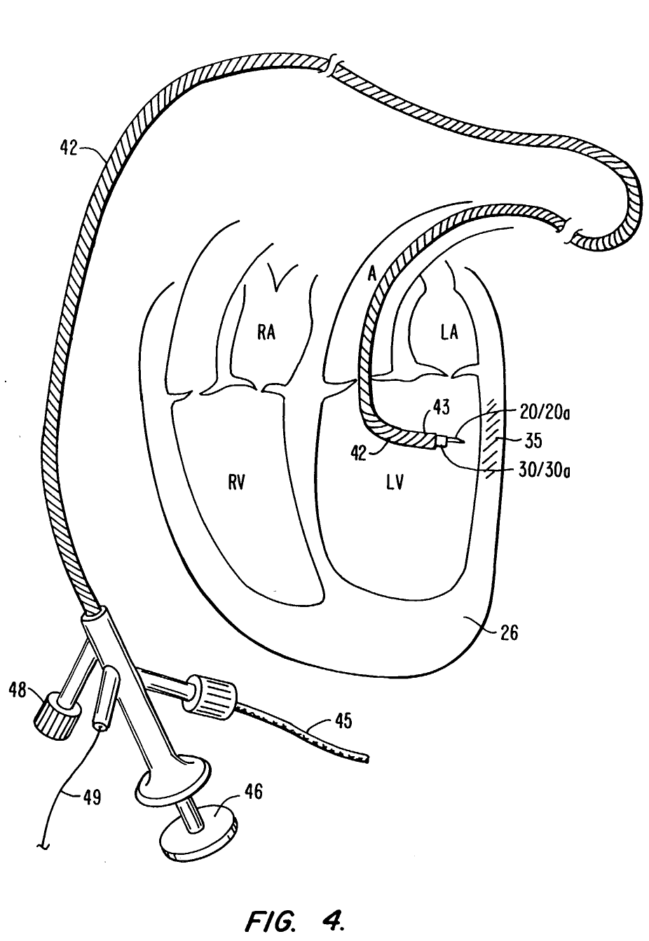

FIG. 4 is a pictorial view of a preferred embodiment of the device of FIG. 3A, 3B, or 3C as inserted into the left ventricle LV of a patient's heart with catheter 42 positioned proximate a diseased region 25 of a patient's myocardium 26. Catheter 42 is preferably provided with a guidewire 45, a distal tip deflection actuator 46 for controlling the position of distal end 43 of the catheter 42. A flush luer 48 is adapted to provide plumbing for contrast dye or for the irrigation of the guidewire lumen. Catheter 42 would preferably be rigid enough to allow for pushability and torqueability as required for typical intracardiac procedures. Electrical connectors 49 are also typically provided for powering emitter 30 and other transducers which will be described herein.

The internal guidewire 45 used to direct the distal tip 43 of catheter 42 would preferably be received in a separate lumen from that of injection needle 20/20a (ie: injection needle 20 or 20a). However, it is to be understood that guidewire 45 may itself comprise injection needle 20 or 20a. Conceivably, however, guidewire 45 and injection needle 20/20a can both be received in the central lumen 44 with guidewire 45 first positioning distal head 43 of catheter 42. Subsequently, guidewire 45 would then be removed such that injection needle 20/20a can be slidably received in central lumen 44 such that injection needle 20/20a passes out of distal end 43 of catheter 42 to a location past vibration energy emitter 30 and into the target tissue.

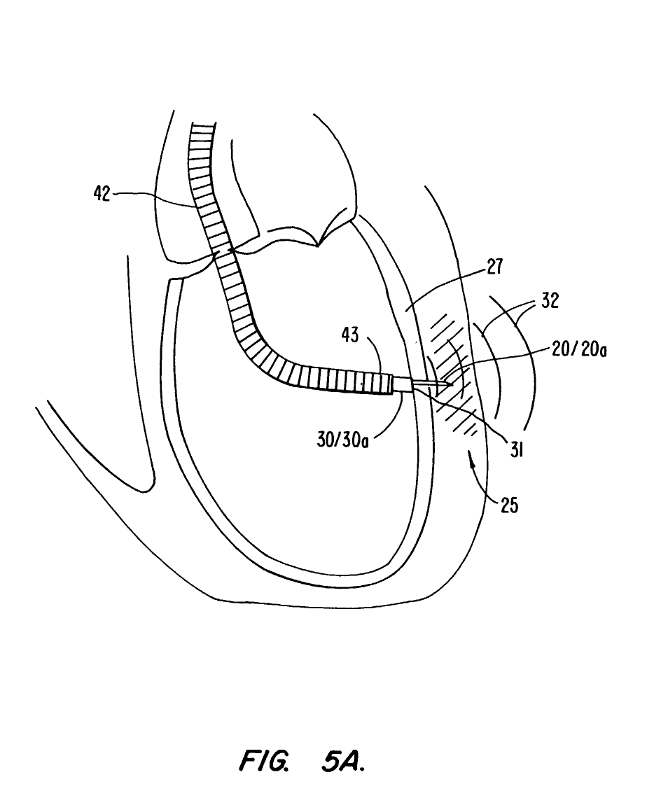

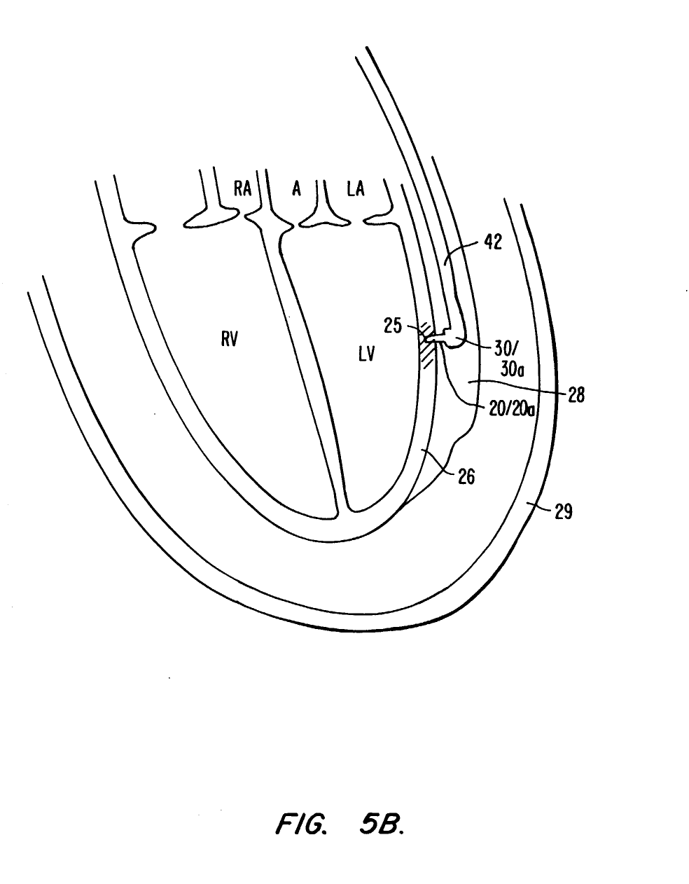

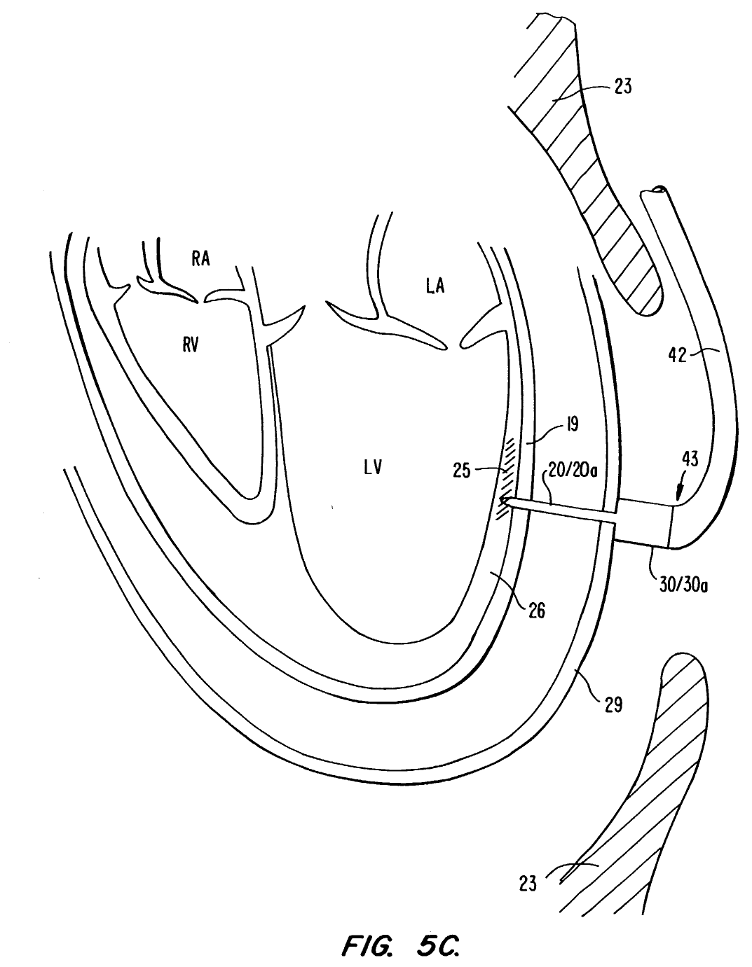

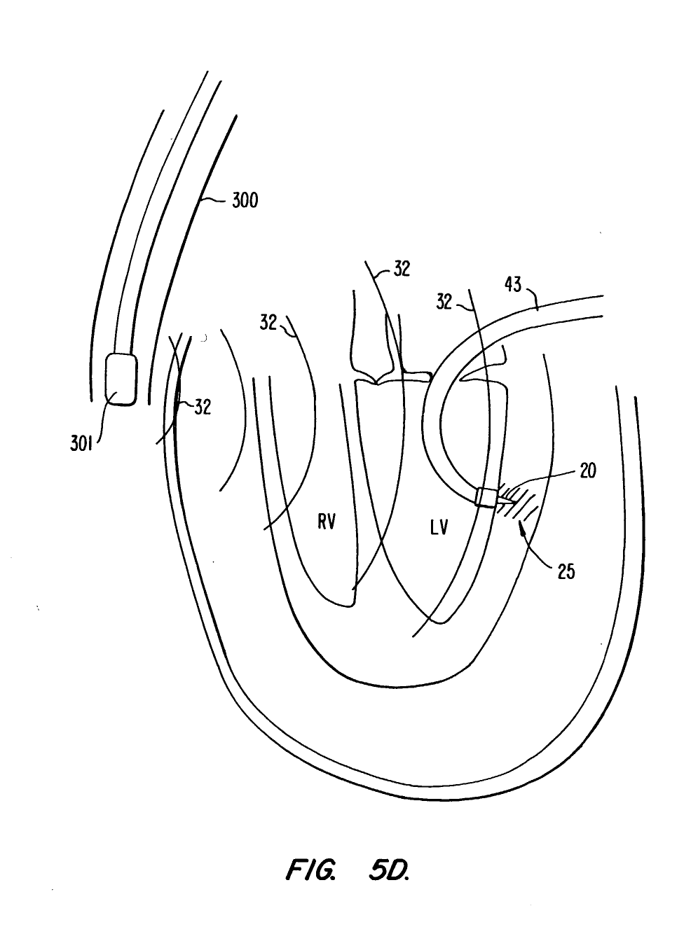

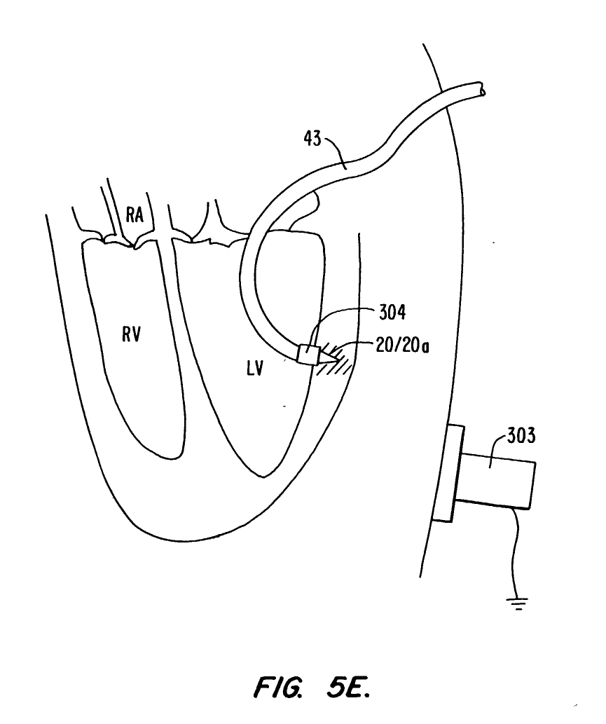

In the preferred method of enhancing cellular absorption of a substance delivered into a patient's myocardial tissue, the first step is the positioning of distal end 43 of catheter 42 proximal diseased tissue region 25. The present invention comprises a variety of different preferred approaches to positioning distal end 43 of catheter 42 into the patient's myocardium. Specifically, FIG. 5A illustrates an approach through the endocardium, FIG. 5B illustrates an approach through a coronary artery, FIG. 5C illustrates an open chest procedure approach through the epicardium, FIG. 5D illustrates an approach wherein the needle and ultrasonic source are separated, wherein the needle approaches through the endocardium with the ultrasonic emission originating from a transesophageal device or a transthoracic device, and FIG. 5E illustrates an approach wherein the needle and ultrasonic source are separated, wherein the needle approaches through the endocardium with the ultrasonic emission originating from an external location on the surface of the patient's skin.

As is shown in FIG. 5A, needle 20/20a is extended through the patient's endocardium 27 to penetrate into diseased region 25 and a fluid suspension containing the substance to be delivered is then passed into target region 25 by injection needle 20/20a. The energizing of transducer 30/30a generates ultrasound waves 32 which cause the tissue in target region 25 to vibrate by an amount sufficient to enhance absorption of the injected substance into this tissue. Preferably, planar vibrational surface 31 will be positioned in flush contact to endocardium 27, thereby providing optimal vibration energy transfer and controlling the penetration depth of injection needle 20/20a.

As is shown in FIG. 5B, access to myocardium 26 can also be achieved with catheter 42 positioned intravascularly in coronary artery or vein 28 with injection needle 20/20a passing through the lumen wall and into myocardium 26.

As is shown in FIG. 5C, access to myocardium 26 can also be achieved with device 42 positioned in or over the patient's pericardium 29 with injection needle 20/20a passing through epicardium 19. This approach may be accomplished in surgical procedures in which access to the patient's heart is achieved either through the sternum or between the ribs 23.

As is shown in FIG. 5D, access to the myocardium is achieved by the system depicted in FIG. 5A. It is to be understood, however, that access to the myocardium could also be achieved by the systems depicted in FIG. 5B or 5C. As depicted in FIG. 5D, the source of ultrasonic emission, however, is separated from the substance delivery system such that the ultrasonic energy may be emitted in the esophagus 300 from a large aperture focused transducer 301 or transducer array on a transesophagal probe. Acoustic waves 32 may be divergent or focussed on a small spot consistent with the resolution of the ultrasonic emitter device.

As is shown in FIG. 5E, access to the myocardium is achieved by the system depicted in FIG. 5A. It is to be understood, however, that access to the myocardium could also be achieved by the systems depicted in FIG. 5B or 5C. As depicted in FIG. 5E, the source of ultrasonic emission, however, is separated from the substance delivery system such that the ultrasonic energy may be emitted from a location on the patient's skin by a transducer 303. A transducer 304 may be positioned at the distal end of the catheter adjacent the injection needle 20/20a, with transducer 304 operating as a receiving transducer, measuring the dose of ultrasound energy received adjacent needle 20/20a.

In the specific case of a transducer or transducer array located in the esophageus, a higher frequency device may preferably be employed, such that a level of beam focussing may be achieved. As such, it may not be necessary to achieve the typically 1-2 millimeter beam profile of diagnostic imaging system; instead, beam profiles on the order of 0.5 to 1.0 cm may be preferred. Operating frequencies in the range of 0.5 to 1.5 MHz from plastic piezoelectric ceramics may also be preferred.

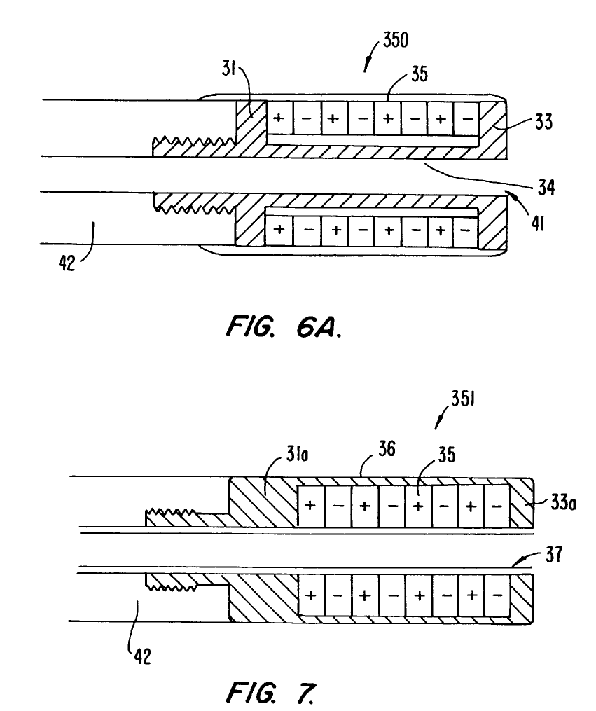

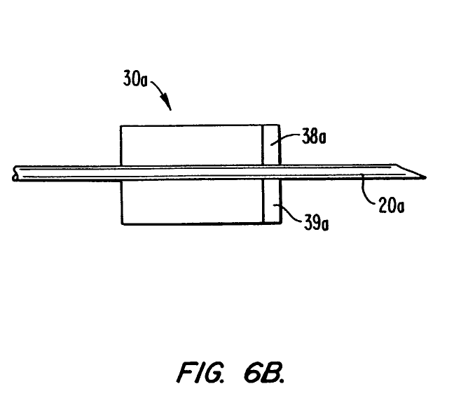

FIG. 6A shows a sectional view of vibration energy emitter 350 similar to emitter 30 shown in FIG. 3A. FIG. 7 shows an embodiment of a vibration emitter 351 in which head mass 33a and inertial mass 31 a are held together by an outer external casing or tensioning skin 36. An internal insulator 37 is provided as a conduit for an injection needle. The systems of FIGS. 6A and 7 are ideally suited for catheter based applications.

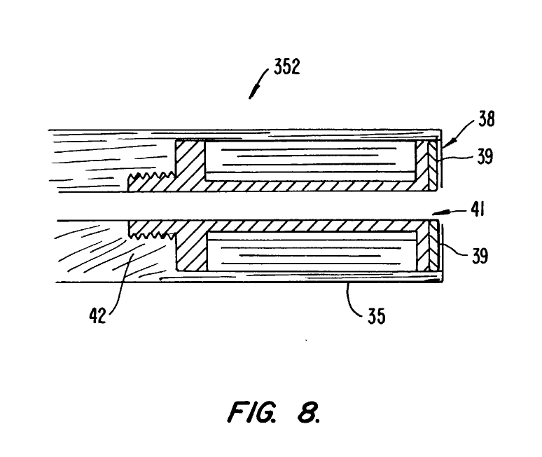

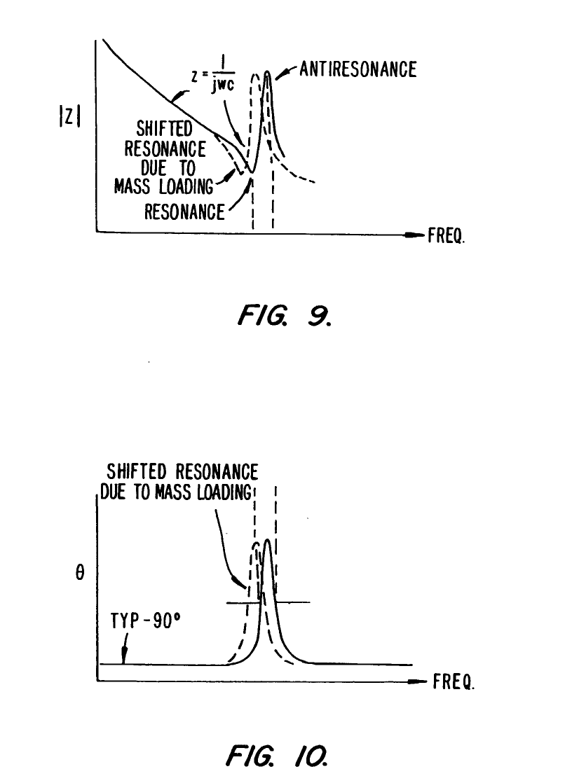

In an alternative embodiment, vibration energy emitter 352, as shown in FIG. 8, further includes an echo ranging transducer 39 which is used to detect contact with the target tissue. Using this embodiment of the present invention in a preferred method, contact with the target tissue is confirmed by observing a change in the impedance of the ranging transducer 39. When ranging transducer 39 contacts the myocardial wall, the additional rigidity of the tissue typically pulls down the resonant frequency of the transducer by as much as 5%. As is illustrated in the electrical impedance plat of FIG. 9, this effect can easily be measured, and it can be used to affirm direct contact between ranging transducer 39 and the myocardial wall. FIG. 10 shows the corresponding impedance phase shift as ranging transducer 39 contacts the myocardial wall. As can be seen, this effect can also be easily measured.

An electrical lead 38, positioned over then distal face of the transducer as shown in FIG. 8, can be used for electrocardiograph monitoring which permits the electrocardiograph function to be traced and mapped onto commercially available electrophysiological equipment such that the location of a specific lesion in the myocardium can be precisely determined, thereby allowing the drug delivery system at the distal end of the catheter to be guided to an optimal location for drug delivery. Alternatively, electrical lead 38 can be in the form of band wrapped around the circumference of the distal end of the catheter. Electrical lead 38 can be used for electrophysiology.

As shown in FIG. 11, ranging transducer 39 can also be used to measure the thickness of the myocardium, as follows. As the tip of catheter 40 approaches the endocardium, ranging transducer 39 is repetitively pulsed in a pulse echo, or A-scan mode. Ultrasound waves 50 will reflect off of the tissue, creating echoes which return to ranging transducer 39. The amplitude and duration of the returning echoes are determined by fluctuations in the acoustic impedance of the tissue and its thickness.

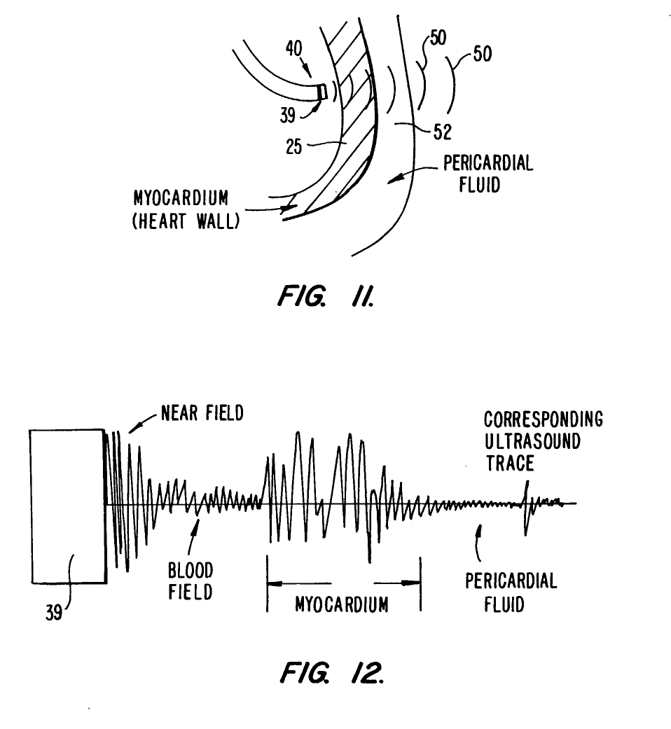

As shown in FIG. 12, which represents the amplitude and duration of the ultrasound echo, the distance from the ranging transducer 39 to the myocardial surface is represented by a low amplitude blood field echo. The myocardium presence is represented by a high amplitude echo, the duration of which is proportional to its thickness, and the pericardial fluid is represented by a low amplitude echo. Accordingly, measurements can be easily made to determine the thickness of the myocardium. The ranging transducer and therapy transducers may be separate piezoelectric ceramic devices, although electrode patterning may allow the use of a single piezoelectric component.

As the operator moves the present device from site to site making multiple injections and applying vibrational energy, the echo ranging transducer 39 would first ascertain whether direct contact has been made with the myocardial wall. Thereafter, transducer 39 could be used to determine the wall thickness such that the proper depth setting for the injection needle plunge could be determined. Doppler signal processing of the A-mode traces 50 might further help delineate the margin. Software may then compute the thickness of the myocardium.

FIG. 6B shows a sectional view of vibration energy emitter 30a as was shown in FIG. 3B, having a ranging transducer 39a and electrical lead 38a positioned thereon, operating similar to ranging transducer 39 and electrical lead 38, described herein.

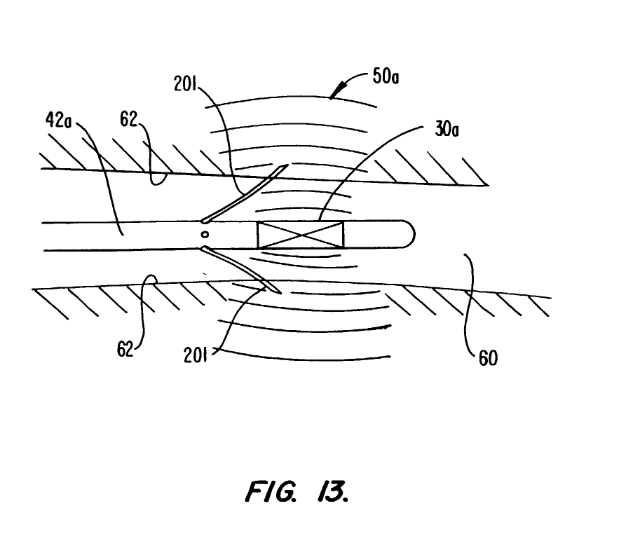

In yet another embodiment of the present device, as illustrated in FIG. 13, catheter 42a is received into a intraluminal cavity 60. Intraluminal cavity 60 can either be a naturally occurring cavity in a patient's body or a cavity formed by injection of a needle into the patient's body. A drug or other substance is delivered into a target region of tissue by puncturing cavity wall 62 of intraluminal cavity 60 by injection needles 201. Preferably, injection needles 201 are disposed to extend radially outward from catheter 42a as shown. In addition, injection needles 20a are preferably retractable into catheter 42a such that in a preferred method, catheter 42a can first be conveniently inserted into lumen 60, and subsequently, injection needles 201 can then be radially extended such that they puncture wall 62 at a variety of radial locations. This radial puncturing of the wall of the interluminal cavity would operate to center catheter 42a within the intraluminal cavity 60. In this embodiment, vibrational energy emitter 30a would emit vibrational energy radially outward as shown by ultrasonic waves 50a. Fluoroscopic imaging may be used to define a luminal diameter and allow the preferred setting of vibrational energy per the observed distance between the catheter 42a to wall 62.

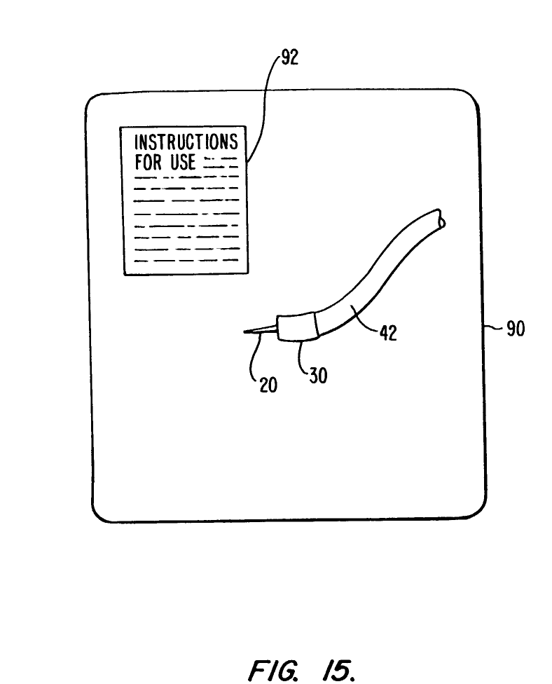

The present invention also includes a kit 90, as seen in FIG. 15, which includes any of the preferred systems for enhancing cellular absorption of a substance as described herein, for example, a catheter 42 having an ultrasonic emitter 30/30a and an injection needle 20/20a, as has been described. Also included in kit 90 are instructions for use 92 which may be in the form of literature accompanying the system, writing on packaging material, information stored on video or audio discs, electromagnetic data storage formats, or other data storage and presentation media. Instructions for use 92 set forth any of the preferred methods described herein.

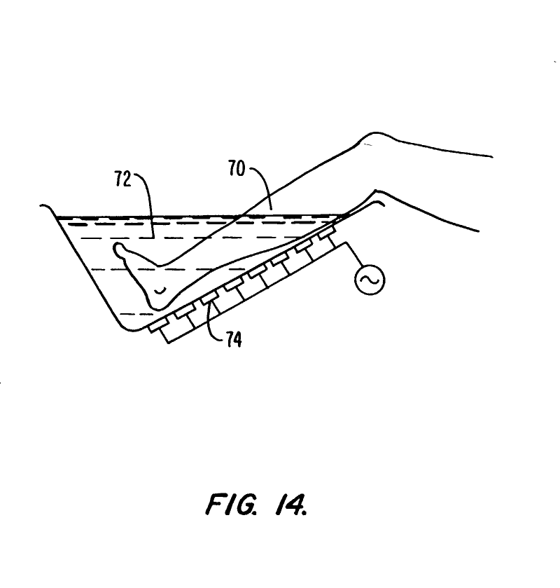

In another aspect of the present invention, as seen in FIG. 14, a patient's leg 70 is received into a fluidic bath 72. A plurality of ultrasonic vibrational energy emitters 74 are provided to subject the fluidic bath to ultrasonic vibrational energy. The apparatus shown in FIG. 14 is particularly useful for patients requiring treatment for ischemia and other vascular problems in the leg, such as may result from cardiovascular disease or diabetes. In a preferred method, which would improve vasculature and reduce pain, a series of multiple injections are typically made in the patients leg from just below the knee to the ankles. The apparatus of FIG. 14 then permits the entire leg of the patient to be subjected to an ultrasonic environment, with the ultrasound vibrations enhancing the cellular absorption of a drug or other substance into the leg. Alternatively, such a treatment method and apparatus may be employed on the patient's arm, hand or foot. An advantage of this apparatus is that a large area of the patient's body can be subjected to ultrasound without the problems of acoustic beam spreading and unwanted amplification, as follows.

Sharply focusing an acoustic beam at a target tissue region substantially amplifies the acoustic power at any point, but then the beam will need to be swept back and forth over the entire surface area to achieve therapeutic levels over a large volume of tissue. This sweeping may require an unacceptable amount of time. To eliminate the need for such sweeping, the acoustic beam might be defocussed to provide acoustic energy over a large volume, at a lower power level.

A fluidic environment will transmit ultrasonic energy more readily than a gaseous environment. Accordingly, with the present invention, the use of fluidic bath 72 will overcome the problem of acoustic beam spreading which would have required the beam to be focused and amplified at any particular location in the leg. As such, the problem of topical administration of ultrasound is overcome.

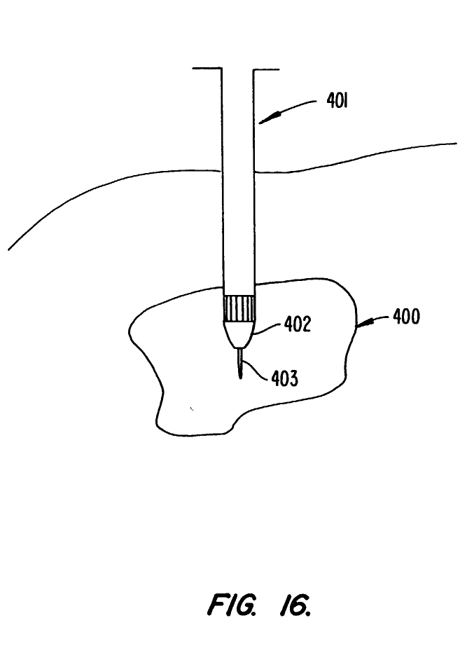

In another aspect of the present invention, needle injection and sonication can be applied in man made lumens within the body, such as those depicted in FIG. 16 for treating soft tissue lesions 400. A semi-rigid tube 401 similar to the catheter configuration previously described is inserted into the subject's body, and directly into the lesion side, by conventional clinical techniques. Semi-rigid tube 401 contains ultrasonic emission surfaces 402 at it's distal tip and an injection needle 403 also protruding from it's distal tip. This technique can be useful for treating typically cancerous lesions of the brain, breast or liver.

The following examples are offered by way of illustration, not by way of limitation.

Experimental

Experiment Number One:

Materials and Methods:

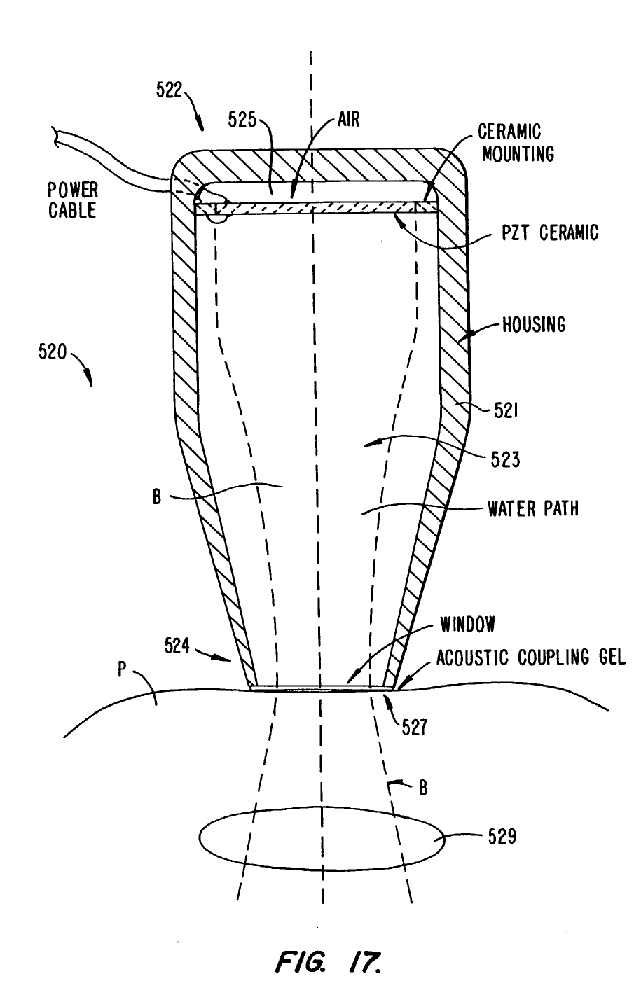

Samples of plasmid DNA (0.5 ml) were injected at a depth of about 4 mm into the thigh muscle of New Zealand white rabbits. The DNA, pCMV-beta-galactosidase, was formulated at 200 mg/ml in saline. The injected sample was found to widely disperse, covering a length of about 4 cm parallel to the muscle fibers and at depths within the tissue which varied from injection to injection. Immediately after the sample injection, an ultrasonic (US) transducer was contacted to the muscle surface and ultrasound energy was applied. There were five overlapping US treatments, each for 1 minute, covering a length of about 3 cm parallel to the muscle fibers. The multiple treatments intended to cover sufficient area assuring the tissue injected with DNA was subjected to the ultrasound. Two different transducer designs were tested. One operated at 1 MHz and produced ultrasound with a beam diameter of about 1 cm (FIG. 17). The other operated at 193 kHz with similar beam characteristics.

The wide beam transducer illustrated in FIG. 17 provides a wide beam ultrasound delivery system which has the advantage of delivering therapeutic ultrasound energy over a large tissue volume such that, in preferred aspects, ultrasound energy can be uniformly distributed over the region in which a therapeutic substance has been injected intramuscularly. An advantage of the present invention is that by distributing a uniform field of ultrasound energy over a large tissue volume, cellular uptake of injected substances such as therapeutic DNA can be substantially enhanced over the entire region in which the injected DNA spreads.

The wide beam ultrasound delivery system of FIG. 17 comprises a housing having an opening at its distal end with an ultrasound transducer suspended within the housing. The ultrasound transducer is positioned in contact with an acoustic couplant material which substantially fills the housing. In the present experiment, the acoustic couplant material was water.

A flexible skin-contact window is disposed across the opening at the distal end of the housing. The skin-contact window was positioned adjacent to the patient's skin such that therapeutic ultrasound energy was conducted from the ultrasound transducer along through the fluid-filled housing and then through the skin-contact window and into the patient.

The housing of the ultrasound delivery system was generally cylindrical and tapers to a narrow distal end which assists in focusing the ultrasound energy emitted by the transducer. Accordingly, the ultrasound energy beam was focused through a narrow region which may be disposed within the housing, or alternatively, the ultrasound energy can be focussed at a transdermal depth.

The experimental ultrasound transducer was generally planar and circular in shape. The transducer of the present invention preferably have a large surface area which may be constructed to range from 1 in³, to 1000 in³.

The fluid which substantially fills the housing of the ultrasound delivery system operates as an acoustic couplant material which transmits the ultrasound energy generated by the transducer therethrough to the skin-contact window and into the patient.

Specifically, FIG. 17 is a sectional side elevation view of the wide aperture beam delivery system. Ultrasound delivery system 520 comprises a housing 521 having a proximal end 522 and a distal end 524. An ultrasound transducer 525 is disposed at the proximal end 522 of housing 521 as shown. Housing 521 was generally cylindrical in shape and was tapered to a narrow distal end 524, as shown. Transducer 525 was made of a ceramic material. Distal end 524 of housing 521 was covered by a flexible skin-contact window 527 which was supported against the skin of patient P. A standard acoustic coupling gel was applied between window 527 and the skin of patient P, to facilitate the transmission of therapeutic ultrasound energy to the patient.

Housing 521 is filled with an acoustically couplant material, which comprised a fluid 523, in this case water. Fluid 523 operated to conduct a beam of ultrasound energy therethrough from transducer 525 to skin-contact window 527. An advantage of substantially filling housing 521 with fluid 523 was that a beam B of ultrasound energy (shown as a dotted line) was passed therethrough as a wide beam of ultrasound energy which can be selectively focussed to pass through a particular therapeutic target focal region 529 at a preferred transdermal depth in the patient.

It was observed that the present wide aperture beam delivery system produced a generally uniform ultrasound field at a transdermal depth of about 2 to 5 cm, and especially at 3 to 4 cm.

An air pocket 528 was provided on one side of transducer 525 such that substantially all of the ultrasound energy emitted by transducer 525 was then directed distally through fluid 523 towards skin-contact window 527 at distal end 524 of housing 521, due to air pocket 528 being an extremely poor conductor of ultrasound energy. Transducer 525 yielding a generally uniform ultrasound beam having a width of at least 0.1 cm, but generally over 0.5 cm, and even over 1.0 cm.

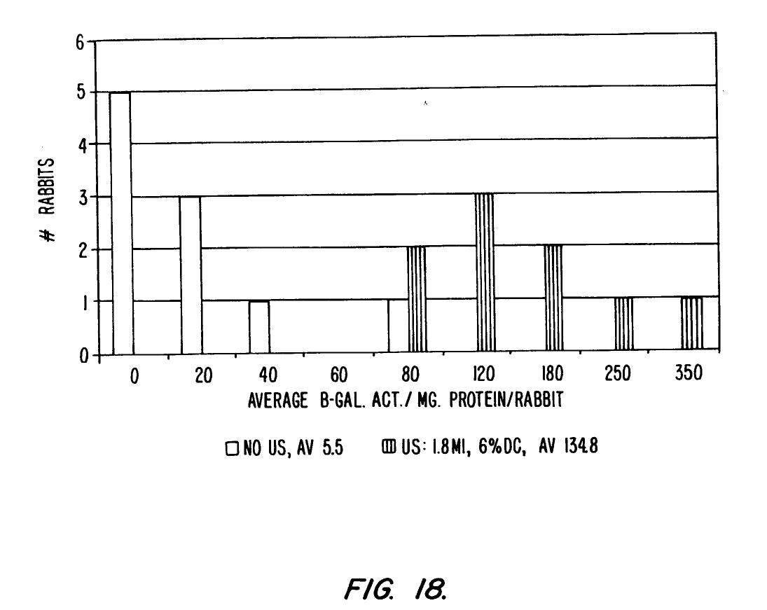

After five days the animals were sacrificed. Each thigh had 9 samples collected in a 3 by 3 array in the area exposed to ultrasound. The muscle samples had dimensions of about 1×1×0.5 cm (W×L×H). Protein was then extracted from the tissue and measured for beta-galactosidase enzyme activity and total protein. Beta-galactosidase activity was normalized to the protein content and expressed as activity per protein mass. For each rabbit thigh, an average beta-galactosidase activity was then calculated from the 9 samples. Tables 1 and 2, and FIG. 18 present the results.

Results:

The results are summarized in Table 1 where “No US” and three US conditions are compared. Expression levels are presented for each treatment comprising the mean beta-galactosidase activity from 9 to 11 rabbits for each group. The ultrasound condition, 1 MHz, 1.8 MI (mechanical index), 6% duty cycle, yielded the best results showing about a 25 fold enhancement of transfection versus the “No US” exposure conditions as set forth below.

TABLE 1
INTRAMUSCULAR GENE DELIVERY:
RESULTS

| | | | Bkgrnd | |
|---|---|---|---|---|
| Treatment | N | B-gal/mg | Crrct. | US/no US |
| No US | 10 | 49.8 +/− 30. | 5.5 | — |
| 1 MHz, 2 MI, 1.5% DC | 11 | 102.3 +/− 103* | 58.0 | 10.5 |
| 1 MHz, 0.5 MI, 25% DC | 9 | 124.0 +/− 81.2** | 79.7 | 14.5 |
| 1 MHz, 1.8 MI, 6% DC | 9 | 179.1 +/− 77.7** | 134.8 | 24.5 |

Background: 44.3
*p = 0.0153
**p = 0.0001

From the same experiment, the distribution of beta-galactosidase expression levels for the individual rabbits was plotted as a histogram in FIG. 18 showing the “No US” and the 1 MHz, 1.8 MI, 6% DC conditions. All 9 of the rabbits treated with this ultrasound condition showed elevations in beta-galactosidase expression.

In the low frequency exposures at 193 kHz with similar transfection conditions, the effect of the ultrasound was studied and results are presented in Table 2. With 193 KHz, 1.09 MI, 1.3% duty cycle about a nine fold increase in beta-galactosidase expression was observed compared to the “No US” conditions.

TABLE 2
INTRAMUSCULAR GENE DELIVERY:
RESULTS

| | | | Bkgrnd | US/ |
|---|---|---|---|---|
| Treatment | N | B-gal/mg | Crrct. | no US |
| No US | 3 | 114.2 +/− 123.9 | 47.6 | — |
| 194 kHz, 1.09 Mi, 1.3% DC | 3 | 526.0 +/− 43.2 | 459.4 | 9.7 |

Background: 66.6

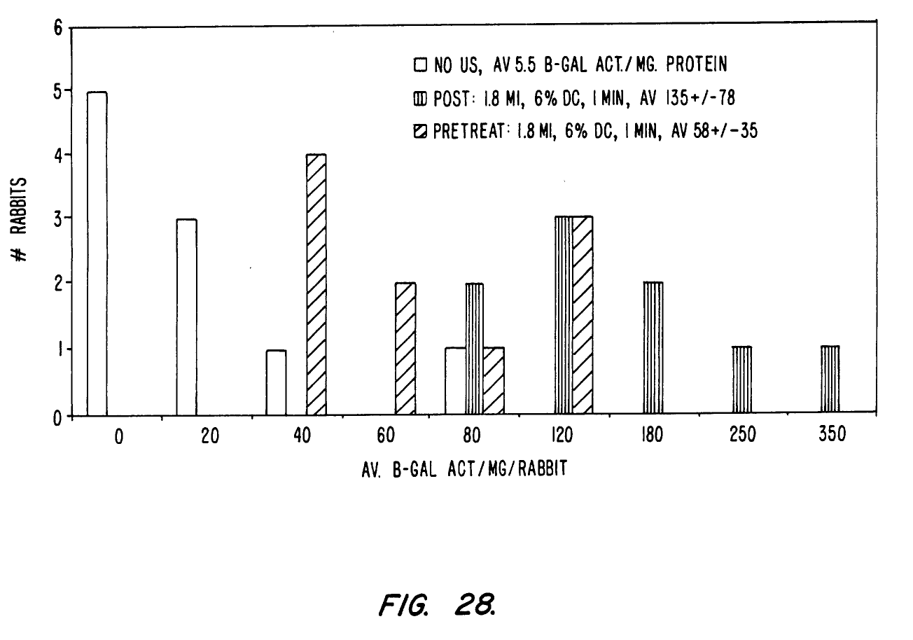

In a second part of this experiment, an ultrasound pre-treatment was applied. Specifically, the above experiment was repeated as set out above with the 5 US exposures carried out at 1 MHz, 1.8 MI, 6% DC conditions, however, the US was applied prior to the VEGF DNA injection. As illustrated in the histogram of FIG. 28, the US pretreatment achieved a 10.5 fold (58/5.5) increase in VEGF transfection, (as compared to the 24.5 fold (135/5.5)increase in VEGF transfection achieved by applying the US after the VEGF injection, as illustrated in Table 1 above and in FIG. 28.

Experiment Number Two:

Materials and Methods:

An ischemic condition was created in one of the hind limbs of each of a group of New Zealand white rabbits by excising their femoral arteries. Ten days thereafter, VEGF DNA was injected into the ischemic muscle and therapeutic ultrasound at 1 MHz, 1.8 MI, and 6% duty cycle was applied for 1 minute in 9 ultrasound exposures along the length of the thigh. At 40 days after the creation of the ischemic condition, a variety of angiogenesis parameters, including blood flow, blood pressure ratio, and angiographic score, were tested.

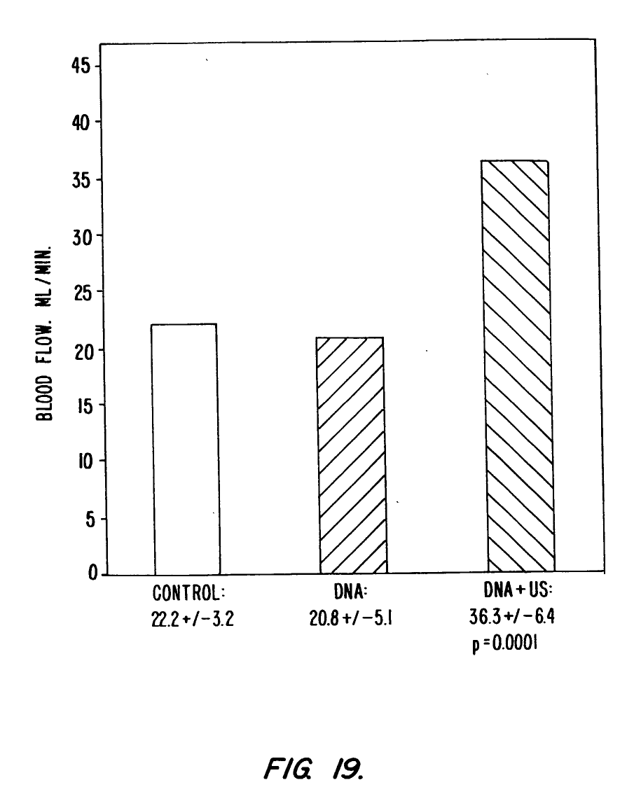

In a first part of this experiment, (illustrated in FIGS. 19, 20, 22, 23, 25, and 26), the VEGF DNA was prepared at a dosage of 100 ug/rabbit and was given to younger rabbits being about 6 months in age.

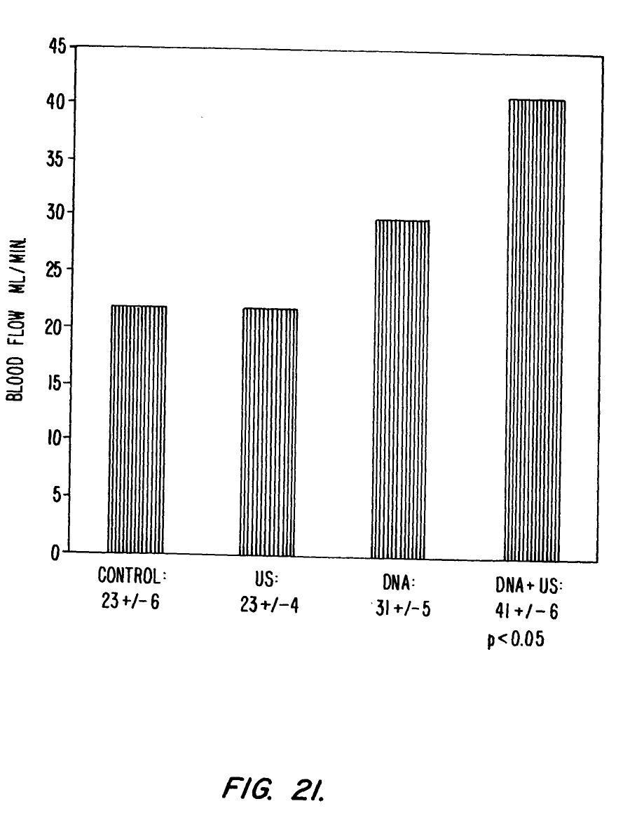

In a second part of the experiment, (illustrated in FIGS. 21, 24 and 27), the VEGF DNA was prepared at a dosage of 500 ug/rabbit, and was given over 5 injections to older rabbits being about 5 years in age.

Older rabbits were selected for the higher dose DNA since age is known to impair the angiogenic effect of VEGF, presenting an additional barrier for ultrasound gene delivery. Therefore, older rabbits were used for the high DNA dose because there was a concern that with young rabbits the higher dose may have produced the maximal angiographic response in the rabbit ischemic hind limbs model making it impossible to detect further angiogenesis when ultrasound was employed. Since older rabbits are angiogenically impaired, they would produce a lower biological response with the high DNA dose alone.

In both the first and second parts of the experiment, ultrasound in the range of 1 MHz, 1.8 MI and 6% duty cycle was applied with the wide beam delivery system illustrated in FIG. 17. Comparisons were made to a rabbit control group and between rabbit groups to which ultrasound was, and was not, applied concurrent with VEGF injection.

Results:

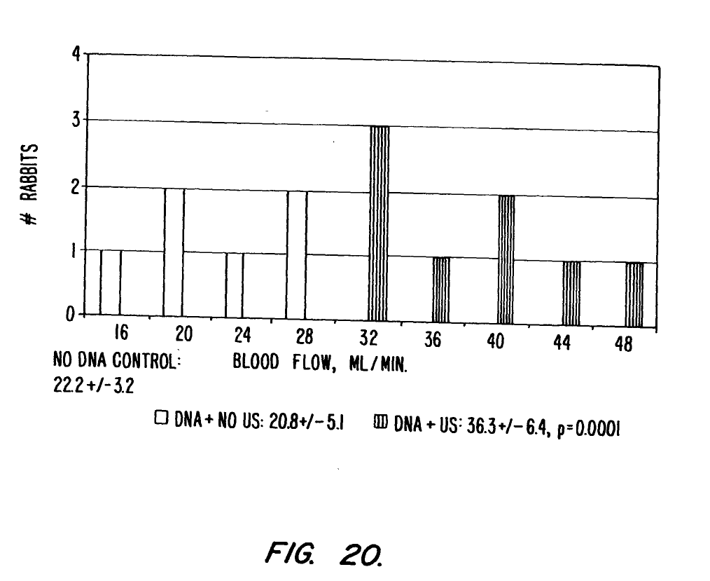

FIG. 19 shows the increased blood flow as measured by a Cardiometrics Doppler wire, from a young rabbit 100 ug VEGF DNA dose control average of 22.2 mL/min to 36.3 mL/min when VEGF DNA was injected concurrent with the application of ultrasound energy. FIG. 20 shows the resulting measurement data for individual rabbits which is presented as an average in FIG. 19.

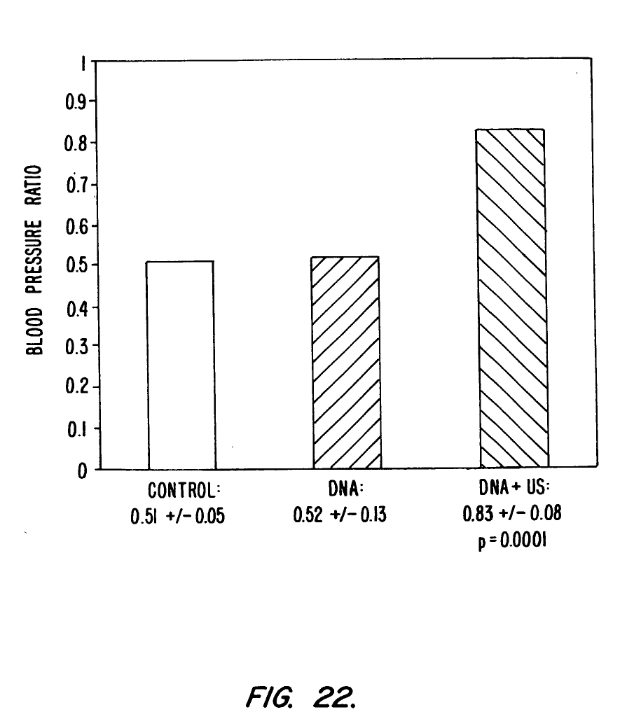

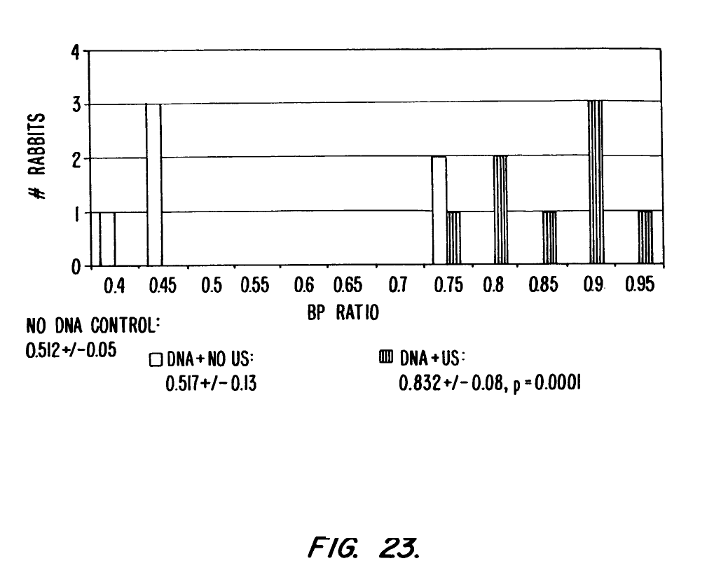

FIG. 22 shows the increased blood pressure ratio comparing ischemic thigh and normal untreated thigh from a young rabbit control group average of 0.512 to 0.832 when VEGF DNA was injected concurrent with the application of ultrasound energy. FIG. 23 shows the resulting measurement data for individual rabbits which is presented as an average in FIG. 22. Lower limb calf blood pressure was measured using a Doppler flowmeter to detect the pulse of the posterior tibial artery and a 2.5 cm wide inflatable cuff was applied over the upper calf to detect the systolic pressure.

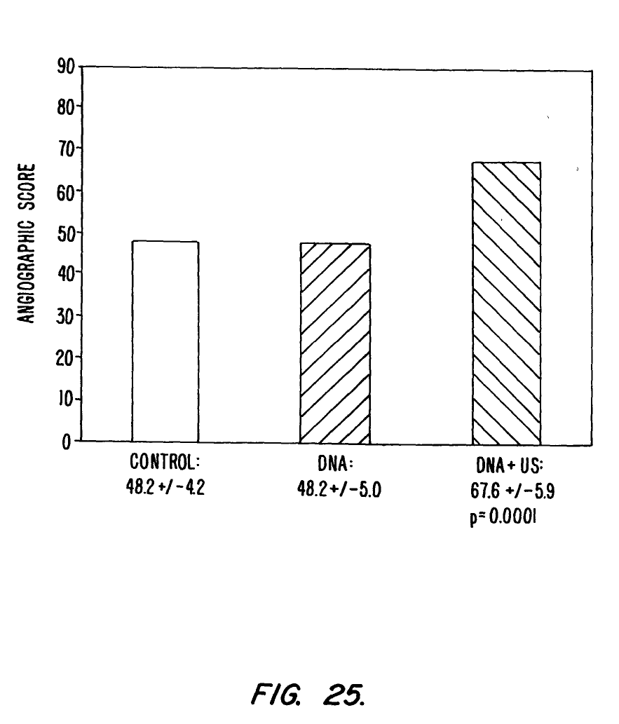

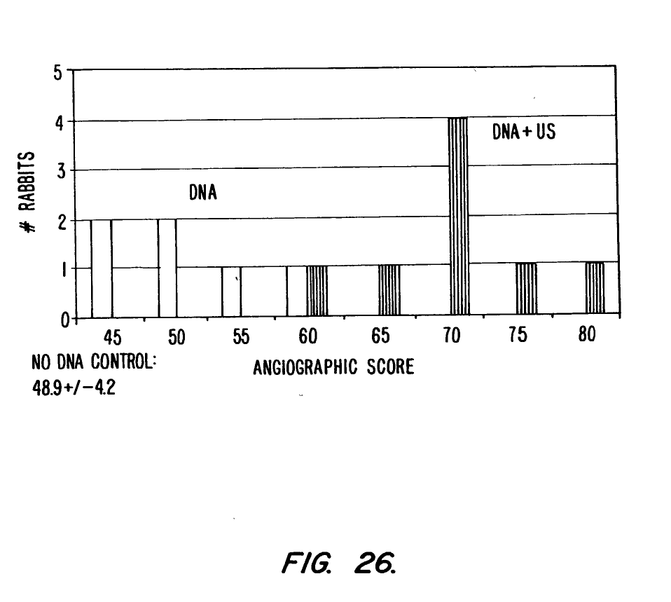

FIG. 25 shows the increased angiographic score from a young rabbit control average of 48.2 to 67.6 when VEGF DNA was injected concurrent with the application of ultrasound energy. FIG. 26 shows the resulting measurement data for individual rabbits which is presented as an average in FIG. 25.

Angiograms were performed with a Medrad angiographic injector delivering contrast media to the internal iliac artery. Angiographic score was determined by overlaying a grid of 2.5 mm circles spaced 5 mm on the angiographic film and counting the number of opacified arteries crossing the circles then dividing by the total number of circles.

FIG. 21 shows the increased blood flow from an older rabbit control average of 23 mL/min to 41 mL/min when VEGF DNA was injected concurrent with the application of ultrasound energy.

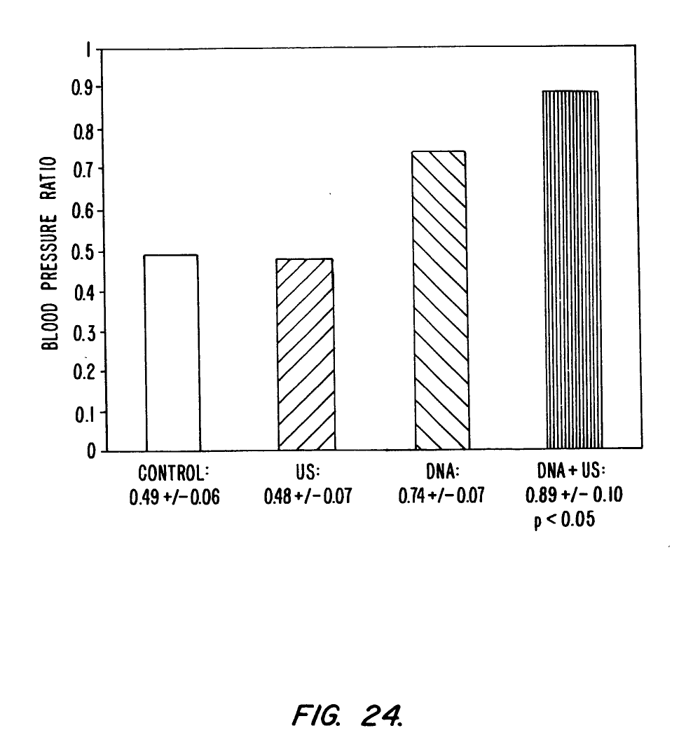

FIG. 24 shows the increased blood pressure ratio from an older rabbit control average of 0.49 to 0.89 when VEGF DNA was injected concurrent with the application of ultrasound energy.

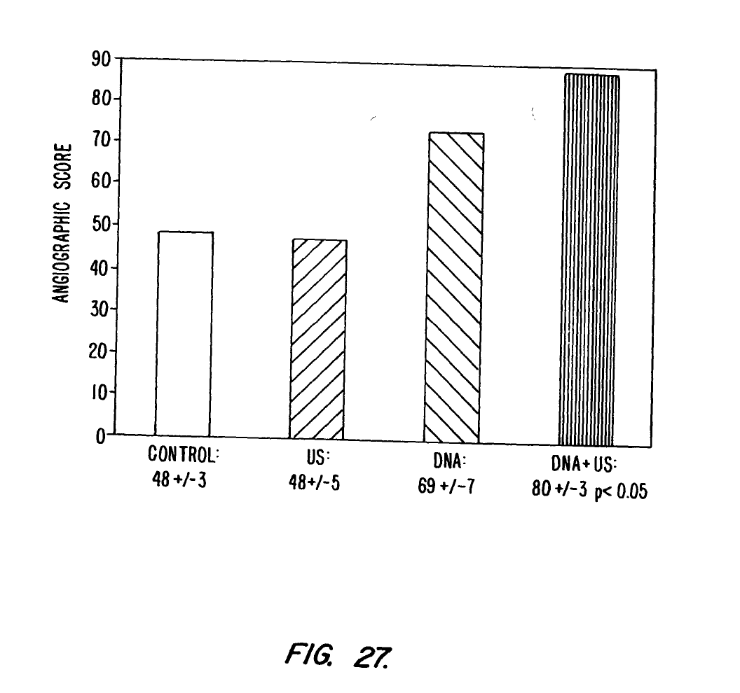

FIG. 27 shows the increased angiographic score from an older rabbit control average of 48 to 80 when VEGF DNA was injected concurrent with the application of ultrasound energy.

As can be seen in FIGS. 19, 22 and 25, blood flow, blood pressure ratio and angiographic score all increase for the younger rabbits when ultrasound energy is applied concurrently with VEGF DNA injection

As can be seen in FIGS. 21, 24, and 27, blood flow, blood pressure ratio and angiographic score all increase for the older rabbits with VEGF DNA injection alone, but all increase to a greater degree when ultrasound energy is applied concurrently with VEGF DNA injection.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A device for enhancing cellular absorption of a substance delivered into a target region of a patient's body, said device comprising:
a housing having an internal cavity with a flexible window at its distal end;
an ultrasound transducer suspended within the internal cavity and spaced proximally of the window; and
an acoustic couplant material substantially filling the cavity between the transducer and the openings
wherein the flexible window is positionable adjacent to a patient's skin to conduct ultrasound energy into the patient.

2. A device as in claim 1, wherein the transducer is planar.

3. A device as in claim 2, wherein the transducer has an area greater than 1 in².

4. A device as in claim 3, wherein the transducer is generally parallel to the window.

5. A device as in claim 1, wherein the housing internal cavity is cylindrical.

6. A device as in claim 5, wherein the internal cavity is tapered in the distal direction to focus ultrasound energy from the transducer through the window.

7. A device as in claim 6, wherein the internal cavity is configured to focus the ultrasonic energy at a depth of from 2 cm to 5 cm beneath the skin surface.

8. A device as in claim 7, wherein the device is configured to focus the ultrasonic energy of a depth of from 3 cm to 4 cm beneath the skin surface.

9. A device as in claim 1, wherein the acoustic couplant material is water.

10. A device as in claim 1, wherein the transducer is backed by air on it's proximal side.

11. A device as in claim 1, wherein the transducer operates at a frequency in the range from 20 kHz to 3 MHz, a mechanical index (MI) in the range from 0.5 to 2, and a duty cycle in the range from 1.5% to 25%.
